(19) United States
(12) Patent Application Publication
(10) Pub. No.: US 2026/0069206 A1
(43) Pub. Date: Mar. 12, 2026

(54) BLOOD COMPONENT ASSAYER

(71) Applicant: DYMSENSE LTD., Rishon LeTsiyon (IL)

(72) Inventors: Dor HELLER ZARBEL, Tel Aviv (IL); Rahav RAVIV, Gedera (IL); Eyal Meron, Rosh Ha’Ayin (IL)

(73) Assignee: DYMSENSE LTD., Rishon LeTsiyon (IL)

(21) Appl. No.: 19/387,156

(22) Filed: Nov. 12, 2025

Related U.S. Application Data
(63) Continuation of application No. PCT/IL2024/050464, filed on May 13, 2024.
(60) Provisional application No. 63/466,288, filed on May 14, 2023.

(51) Int. Cl. A61B 5/00; A61B 5/1455

(52) U.S. Cl. CPC A61B 5/4845; A61B 5/1455; A61B 5/6826; A61B 2562/185

(57) ABSTRACT

Apparatus for assaying blood alcohol concentration (BAC), the apparatus comprising: a housing for receiving a finger of a person; at least one light source that transmits light at at least one assay wavelength in respective directions so that the transmitted light from all the light sources converges to overlap in a same convergence region that illuminates an assay region of a finger received by the housing; a light sensor positioned to receive and generate an assay signal responsive to assay light reflected by tissue in the assay region; and a light shield that enables assay light from the convergence region to reach the light sensor and shields the light sensor from stray light that is not reflected by tissue in the assay region from assay light originating from the at least one light source.

### RELATED APPLICATION

The present application claims the benefit under 35 U.S.C. 119 (e) of U.S. Provisional Application 63/466,288 filed on May 14, 2023, the disclosure of which is incorporated herein in its entirety by reference.

### FIELD

Embodiments of the disclosure relate to methods and systems for assaying a blood component of a person.

### BACKGROUND

Substance abuse is a major contributing factor in causing road accidents. In particular, alcohol abuse, because of the ease with which alcohol may be acquired, its popular acceptance, and frequent consumption by most segments of the population, contributes significantly to traffic accidents and is a contributing factor in almost a third of all traffic deaths in the US. For example, in 2020 in the U.S. 11,654 people died in drunk driving crashes—about one person every 45 minutes! And whereas it is commonly assumed that relatively low levels of blood alcohol concentration (BAC), which might for example result from social drinking, do not significantly impair a driver's driving performance, even low levels of BAC have been found to reduce driving competence. BACs in a range from about 0.01 to about 0.07 grams per deciliter (g/dl) were indicated as a contributing factor in the traffic deaths of over 2,000 people in the US in 2020.

Accurate and easy to use methods and apparatus for measuring blood components of person that might affect the person's competence to carry out a task would be advantageous. Technology for rapidly and accurately determining a person's BAC prior to allowing the person to take the driving wheel of a vehicle would appear to be particularly advantageous to controlling substance abuse related traffic accidents.

### SUMMARY

An aspect of an embodiment of the disclosure relates to providing methods and systems for relatively quickly and relatively accurately assaying a person's BAC by measuring reflectance of light from the person's finger at a plurality of different wavelengths, hereinafter referred to as assay wavelengths. Light at an assay wavelength may be referred to as assay light.

The assay is performed by a relatively rapid and accurate assayer (RACAS, or RACAS assayer) optionally comprising a contact substrate transparent to light at the assay wavelengths on which a person having BAC assayed presses a finger to establish a contact interface between a surface of the substrate and skin of the finger. RACAS comprises at least one light source controllable to transmit light at each of the assay wavelengths into the contact plate so that transmitted assay light is incident on and is reflected from the contact interface. For each assay wavelength RACAS comprises at least one light sensor optionally coupled to the contact substrate for receiving light reflected from the assay light transmitted into the substrate by the at least one light source from a region of tissue, optionally referred to as assay tissue, in the finger in the vicinity of the contact interface. The at least one sensor generates a signal, hereinafter also referred to as an assay signal, responsive to intensity of the reflected assay light that the at least one sensor receives.

The intensity of the reflected assay light received for each of the assay wavelengths by the at least one sensor and thereby the associated assay signals are functions of the respective intensities and angles of incidence of the assay light incident on the contact interface, minus that portion of the incident assay light that is transmitted into and absorbed by assay tissue of the finger at the contact interface. The absorption is a function of molecular components of the finger tissue, such as by way of example water and hemoglobin, and alcohol present in the assay tissue that absorb and/or scatter the light. RACAS processes the assay signals generated by the at least one sensor to determine concentrations of alcohol and other components, collectively assay components, in the finger assay tissue that absorbed assay light incident on the contact interface and may have affected the assay signals. The determined concentration of alcohol is used to provide a measurement of the person's BAC,

In an embodiment processing the assay signals to determine concentrations of assay components comprises determining concentrations of alcohol and at least one other assay component which maximize a probability function that provides a probability that a given set of assay signals is generated by a particular set of concentrations of the assay components.

In an embodiment the contact substrate comprises a planar plate to which the finger is pressed. Optionally, the contact substrate comprises a curved surface, which may be a portion of a spherical surface, to which the finger is pressed. As pressure at which the finger is pressed to the contact substrate may affect the contact interface, RACAS comprises a pressure sensor that determines the “finger pressure”. RACAS activates transmission and reception of assay light to measure a person's BAC when the pressure sensor indicates that the finger pressure is advantageous for performing the measurement. Optionally, RACAS comprises an indicator light, which responsive to measurements provided by the pressure sensor, indicates to the person when he or she is exerting pressure on the contact substrate advantageous for preforming the BAC measurement.

In an embodiment a RACAS comprises a light shield that protects the at least one light sensor from receiving light, hereinafter also referred to as stray light, that is not reflected by assay tissue from assay light originating from the at least one light source. Optionally, the light shield comprises an optionally collimating shield housing, having an optical entry port facing a region at which assay tissue of a person's finger is located on or in the RACAS during a BAC assay. Assay light entering the light shield through the optical entry port is directed by the collimating action of the light shield and/or by an optical element housed in the light shield to the light sensor.

Optionally, a RACAS may be absent a contact substrate and have no material through which assay light propagates to reach an aperture of the at least one light sensor from the assay tissue. Optionally a RACAS comprises a relatively thin protective panel that is located between an aperture of the at least one light sensor and the assay tissue. In an embodiment the protective panel may function as a contact panel on which a person having the person's BAC assayed by the RACAS places a finger to provide the RACAS with assay tissue for illumination with assay light.

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter.

### BRIEF DESCRIPTION OF THE FIGURES

Non-limiting examples of embodiments of the disclosure are described below with reference to figures attached hereto that are listed following this paragraph. Identical features that appear in more than one figure are generally labeled with a same label in all the figures in which they appear. A label labeling an icon representing a given feature in a figure of an embodiment of the disclosure may be used to reference the given feature. Dimensions of features shown in the figures are chosen for convenience and clarity of presentation and are not necessarily shown to scale

FIGS. 1A and 1B respectively show schematic perspective top and bottom views of a RACAS assayer comprising a substantially hemispherical contact substrate in accordance with an embodiment of the disclosure;

FIG. 1C shows a schematic cross section of RACAS shown in FIGS. 1A and 1B, in accordance with an embodiment of the disclosure;

FIGS. 2A and 2B schematically show a cross section view and a perspective view of a finger of a person's finger pressed to the contact substrate of RACAS shown in in FIGS. 1A-1C, in accordance with an embodiment of the disclosure;

FIG. 3 shows absorptivity spectra for water and alcohol

FIGS. 4A and 4B respectively show schematic views from above and below viewpoints of a RACAS assayer comprising a rectangular parallelepiped contact substrate, in accordance with an embodiment of the disclosure;

FIGS. 5A and 5B respectively show schematic views from above and below viewpoints of a RACAS assayer comprising a contact substrate having a curved contact surface on which a user presses a finger to establish a contact interface, in accordance with an embodiment of the disclosure;

FIG. 6 schematically shows a RACAS comprising a light shield for protecting a light sensor from stray light, in accordance with an embodiment of the disclosure;

FIGS. 7A and 7B schematically show cross-section and perspective views respectively of another RACAS, in accordance with an embodiment of the disclosure;

FIG. 8 schematically shows yet another RACAS comprising a shielded light sensor, in accordance with an embodiment of the disclosure; and

FIG. 9 shows a schematic of a light source comprising a configuration of a plurality of light emitting elements that emit assay light, in accordance with an embodiment of the disclosure.

### DETAILED DESCRIPTION

In the discussion, unless otherwise stated, adjectives such as “substantially” and “about” modifying a condition or relationship characteristic of a feature or features of an embodiment of the disclosure, are understood to mean that the condition or characteristic is defined to within tolerances that are acceptable for operation of the embodiment in an application for which it is intended. Wherever a general term in the disclosure is illustrated by reference to an example instance or a list of example instances, the instance or instances referred to, are by way of non-limiting example instances of the general term, and the general term is not intended to be limited to the specific example instance or instances referred to. The phrase “in an embodiment”, whether or not associated with a permissive, such as “may”, “optionally”, or “by way of example”, is used to introduce for consideration an example, but not necessarily required, configuration of possible embodiments of the disclosure. Each of the verbs, “comprise” “include” and “have”, and conjugates thereof, are used to indicate that the object or objects of the verb are not necessarily a complete listing of components, elements or parts of the subject or subjects of the verb. Unless otherwise indicated, the word “or” in the description and claims is considered to be the inclusive “or” rather than the exclusive or, and indicates at least one of, or any combination of more than one of items it conjoins.

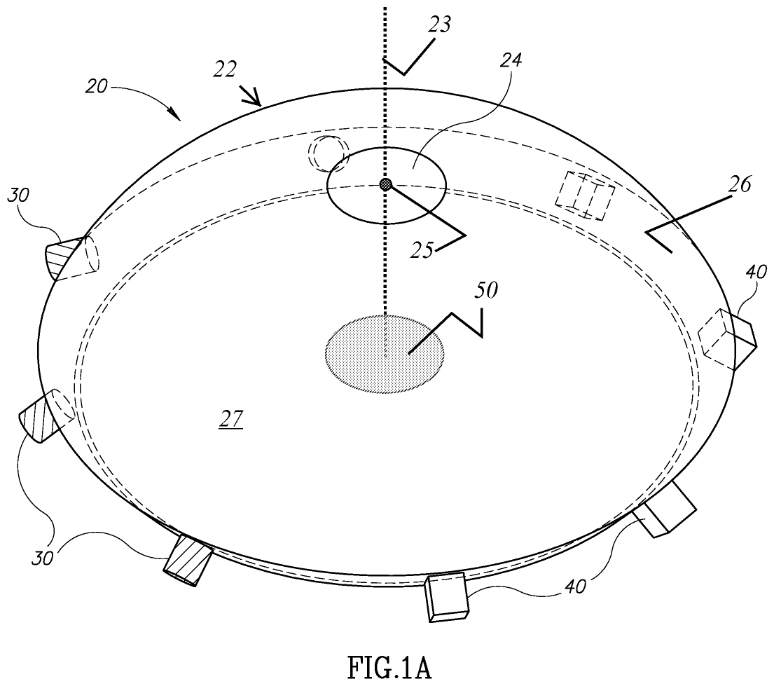

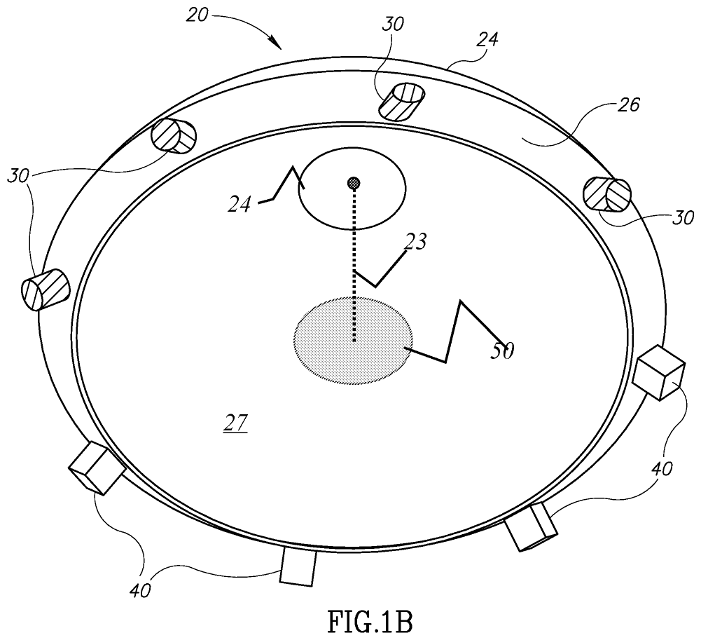

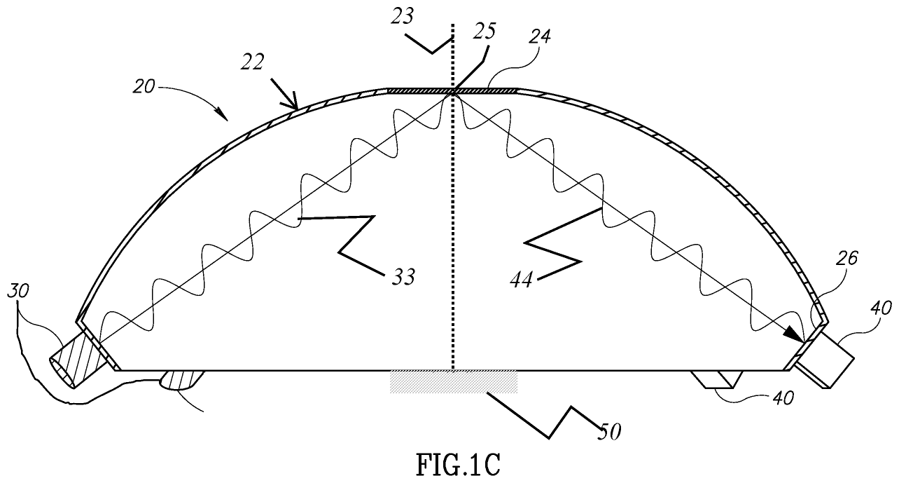

FIGS. 1A and 1B respectively show perspective top and bottom schematic views of a RACAS assayer 20 optionally comprising a substantially hemispherical contact substrate 22 having an axis of rotation 23, in accordance with an embodiment of the disclosure. Contact substrate 22 optionally comprises a planar contact surface 24 and a bevelled edge surface 26 optionally surrounding a planar bottom surface 27 of contact substrate 22 that is perpendicular to axis 23. Contact surface 24 is a region of contact substrate 22 on which a person having his or her BAC determined using RACAS assayer 20 presses a finger. Bevelled edge surface 26 is a surface to which a plurality of light sources 30 and a plurality of light sensors 40 are optically coupled to contact substrate 22. Light sources 30 are configured to transmit assay light so that the transmitted assay light is incident on contact surface 24. Light sensors 40 are configured to sense and generate assay signals responsive to assay light reflected by the contact interface from the incident light.

At least one, optionally piezoelectric pressure sensor 50 is mounted to surface 27, optionally at a center of the surface directly opposite contact surface 24. Pressure sensor 50 generates pressure signals responsive to pressure between contact substrate 22 and a housing (not shown) that houses the substrate generated by force with which a person presses a finger to contact surface 24.

In an embodiment a number of the plurality of light sources 30 is equal to a number of the plurality of light sensors. Optionally, each light source 30 transmits assay light at a different assay wavelength and is paired with a different light sensor 40 that receives and is sensitive to the assay light transmitted by the light source. Paired light sources 30 and sensors 40 are mounted to bevel surface 26 directly opposite each other at respective ends of a diameter of bottom surface 27 so that a central ray of assay light transmitted by a light source 30 that is incident on and reflected from a center 25 of contact surface 24 is incident substantially at a center (not shown) of an aperture of the sensor 40 paired with the light source.

FIG. 1C shows a schematic cross section of RACAS 20 that illustrates a light source 30 transmitting a beam 33 of assay light that is incident on and reflected from center 25 of contact surface 24 as a reflected beam 44. Reflected beam 44 is incident on sensor 40 that is paired with light source 30.

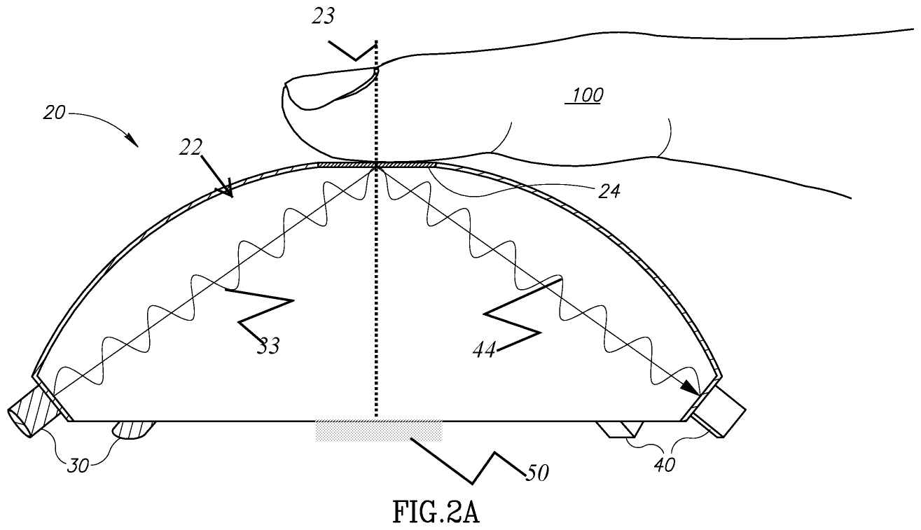

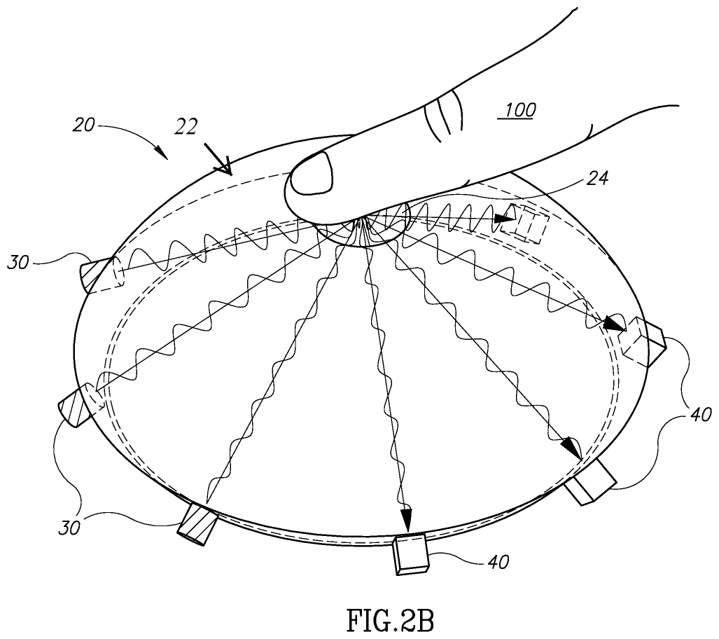

FIGS. 2A and 2B show respective cross section and perspective views illustrating a person (not shown) using RACAS 20 to provide a value for the person's BAC in accordance with an embodiment of the disclosure. The figures schematically show the person pressing a finger 100 to contact surface 24 of substrate 22 to create a contact interface between contact surface 24 and the finger that affects reflection of assay light from the contact surface and thereby assay signals used to determine the BAC that sensors 40 generate responsive to the reflected assay light in accordance with an embodiment of the disclosure. FIG. 2A schematically shows a light source 30 transmitting assay light 33 to contact surface 24 and a sensor 40 paired with light source receiving assay light 44 reflected by contact surface 24 from transmitted light 33. The perspective view of FIG. 2B schematically shows assay light being transmitted to contact surface 24 by all light sources 30 and being received by light sensors 40 respectively paired with the light sources.

It is noted that whereas FIG. 2B appears to show all light sources 30 operating simultaneously to transmit assay light, practice of an embodiment of the invention is not limited to simultaneous activation of all the light sources. Light sources may for example be individually activated sequentially in any order or different groups of light sources may be activated sequentially.

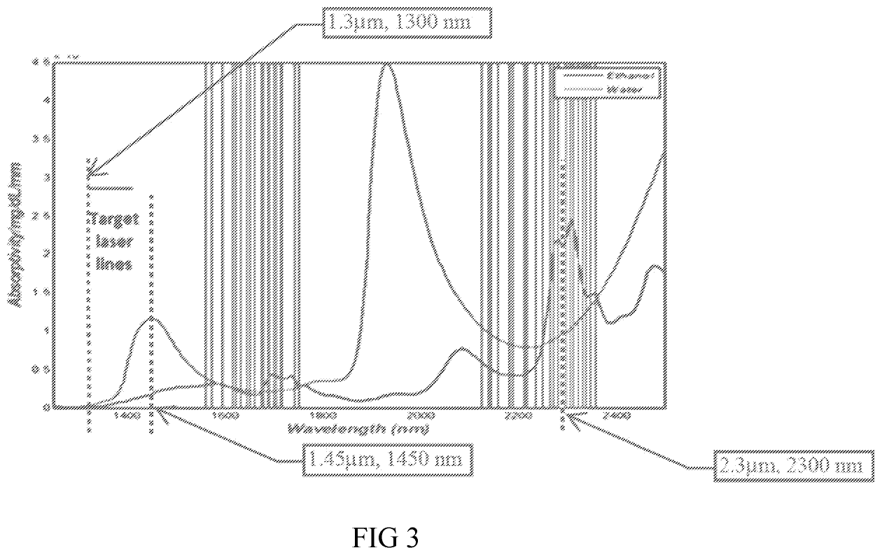

Advantageously, assay wavelengths at which assay light is transmitted by light sources 30 in RACAS 20 include wavelengths for which absorptivity for components of interest in tissue and blood in finger 100 may be used to distinguish concentrations of the components in the finger. In general, such wavelength are wavelengths for which the absorptivities are substantially different. For example, components of particular interest when carrying out a BAC measurement in accordance with an embodiment of the disclosure are of course alcohol and also water. FIG. 3 shows absorptivity spectra for alcohol and water per mg (milligram) per dL (deciliter) per mm. From the absorptivity spectra it appears that advantageous assay wavelengths are wavelengths in wavelength bands comprising wavelengths 1.3 μm, 1.45 μm, and 2.3 μm.

In an embodiment, processing the assay signals generated for example by sensors 40 in FIG. 2B, to determine concentrations of assay components and BAC comprises determining concentrations of alcohol and at least one other assay component that maximize a probability function which provides a probability that a given set of assay signals results from a particular set of concentrations of the assay components.

Assume by way of example that a RACAS in accordance with an embodiment of the disclosure similar to RACAS 20, comprises a plurality of J assay light sources 30 respectively paired with J assay light sensors 40 and that each light source transmits assay light at a different assay wavelength λⱼ (1≤j≤J). Assume further that there are N components of interest, one of which is alcohol, in finger 100 having unknown concentrations ρₙ (1≤n≤N) that may affect an assay signal that a j-th (1≤j≤J) light sensor generates responsive to reflected assay light of wavelength λⱼ incident on the sensor. Let r(c, λⱼ)ₙ for a given skin color, c, represent reflectance for light at assay wavelength λⱼ of the nₜₕ assay component of interest in finger 100. If the assay signal generated by the j-th light sensor 40 responsive to incident assay light at wavelength λⱼ reflected by contact surface 24 to the sensor is represented by R(λⱼ, c, p) then R(λⱼ, c, ρ)ⱼ may be written,

$$R {( {λ}_{j} , c , ρ )}_{j} = \text{} {DS ( {λ}_{j} )}_{j} [ \text{} r {( c , {λ}_{j} )}_{1 ρ1} + r {( c , {λ}_{j} )}_{2 ρ2} + r {( c , {λ}_{j} )}_{3 ρ3} + … r {( c , {λ}_{j} )}_{N} {ρ}_{N} ] \qquad ( 1 )$$

In expression (1) ρ represents the set of concentrations {ρₙ|(1≤n≤N)}, DS(λⱼ)ⱼ is a proportionality constant that is a function inter alia of intensity of light transmitted by the j-th light source, sensitivity of the j-th sensor to light at wavelength λⱼ, and relevant geometric parameters, such as angle of reflection of reflected assay light.

Let a probability density function for the j-th light sensor 40 providing a given expected assay signal R(λⱼ, c, ρ); for known values c* and ρ* of c and p respectively be represented by: [symbol] (R(λⱼ, c, ρ)ⱼ|R*(λⱼ, c*, ρ*)ⱼ), where R*(λⱼ, c*, ρ*); is an expected assay signal for c* and ρ*.

For convenience of presentation, let an actual assay signal R(λⱼ, c, p) j provided by the j-th sensor 40 for unknown concentrations ρ and skin color c be represented by the “short-hand”, Rⱼ, and the set of actual assay signals provided by all J sensors 40 for the unknown values of c and ρ be represented by a vector \vec{R}={Rⱼ|(1≤j≤J)}. In an embodiment, the probability density function for the J sensors 40 providing a given set of actual assay signals \vec{R} for c* and ρ* is assumed to be a multivariate Gaussian density function [symbol] (\vec{R}(λ, c, ρ)) where λ represents the set of assay wavelengths {λⱼ|(1≤j≤J)}. Then [symbol] (\vec{R}(λ, c, ρ)) may be written

$$( \vec{R} ( λ , c , ρ ) ) = ( \frac{1}{{2 π}_{} N / {2}_{( {σ}_{1} {σ}_{2} … {σ}_{N}}} ) exp - \qquad ( 2 )$$

$$[ \frac{{{( {R}_{1} - R * ( {λ}_{1} , c * , ρ \text{*)}}_{1} )}^{2}}{{σ}_{1}^{2}} + \text{} \frac{{{( {R}_{2} - R * ( {λ}_{2} , c * , ρ \text{*)}}_{2} )}^{2}}{{σ}_{2}^{2}} … + \frac{{{( {R}_{N} - R * ( {λ}_{N} , c * , ρ \text{*)}}_{N} )}^{2}}{{σ}_{N}^{2}} ]$$

where it has been assumed for convenience of presentation that a covariance matrix for R* is diagonal. It is noted that in equation (2) the expected assay signals R*, for skin color c* and the concentrations of the assay components ρ* are unknowns to be solved for, and that the Rj are known actual assay signals. Values for c*, ρ*, may be determined by determining which values c*, ρ*, maximize [symbol] (\vec{R}(λ, c, ρ)) for the actual assay signals \vec{R}. Determining the maximizing values may be accomplished using any suitable procedure known in the art. For example α*, ρ*, may be determined using a least squares method or a continuous optimization technique, optionally, a gradient descent.

In an embodiment, assuming that concentration ρ*₁ of the set of concentrations ρ*={ρ*ₙ|(1≤n≤N)} is concentration of alcohol, ρ*₁ is used to determine BAC.

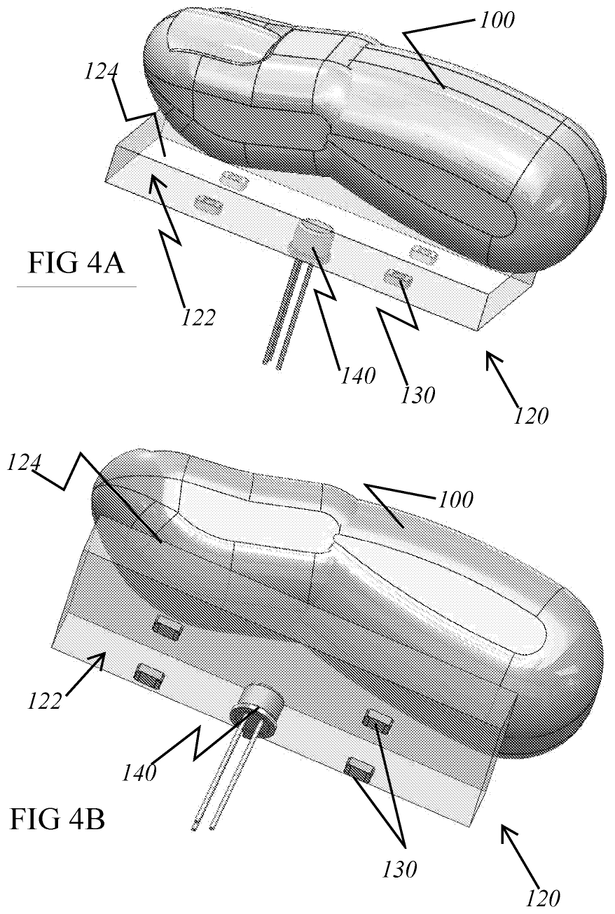

In an embodiment the vector of actual assay signals \vec{R} may be processed by any of various types of artificial intelligences (AI), such as machine learning algorithms, decision trees, regression algorithms, and various types of neural networks. For example, the vector of actual assay signals \vec{R} may be processes by convolutional neural network (CNN) or deep neural network (DNN) to provide a probability for each of a predetermined plurality of BAC ranges that the vector of actual assay signals is a result of a BAC lying in the range.

Whereas in the above example, RACAS 20 comprises a substantially hemispherical contact substrate, practice of embodiments of the disclosure are not limited to hemispherical or spherical shapes. For example, FIGS. 4A and 4B respectively show schematic views from above and below viewpoints of a RACAS 120, in accordance with an embodiment of the disclosure. RACAS 120 comprises a rectangular parallelepiped contact substrate 122 having a planar contact surface 124, to which a person presses a finger 100 to provide a contact interface with the contact substrate and determine the person's BAC, in accordance with an embodiment of the disclosure. RACAS 120 optionally comprises a plurality of, optionally four, light sources 130, each transmitting light at at least one different assay wavelength into substrate 122 and a single light sensor 140 that generates assay signals responsive to incident assay light transmitted by each of light sources 130. Light sensor 140 may comprise a plurality of light sensitive pixels (not shown), each of which generates assay signals responsive to incident assay light transmitted by light sources 130.

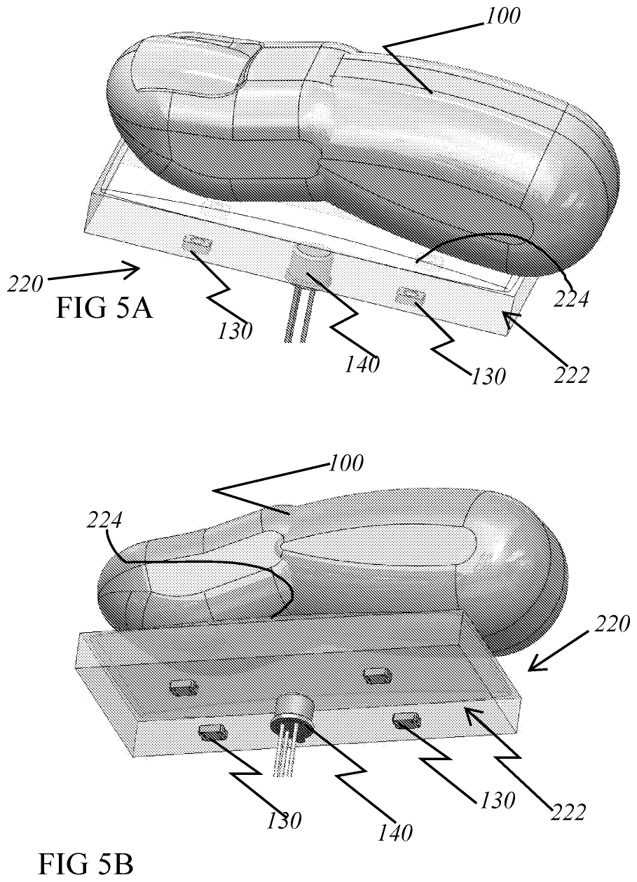

FIGS. 5A and 5B respectively show schematic views from above and below viewpoints of a RACAS 220 similar to RACA 120 and comprising plurality of optionally four light sources 130 and a single light sensor 140, in accordance with an embodiment of the disclosure. However, unlike RACAS 120, RACAS 220 comprises a prism shaped contact substrate 222 having a curved contact surface 224 rather than the planar contact surface 124 of RACAS 120, to which a person presses a finger 100 to determine the person's BAC, in accordance with an embodiment of the disclosure. Curved contract surface 124 has a radius of curvature advantageous for conforming to a person's finger 100 to facilitate providing a relatively large area contact interface between contact substrate 222 and the finger.

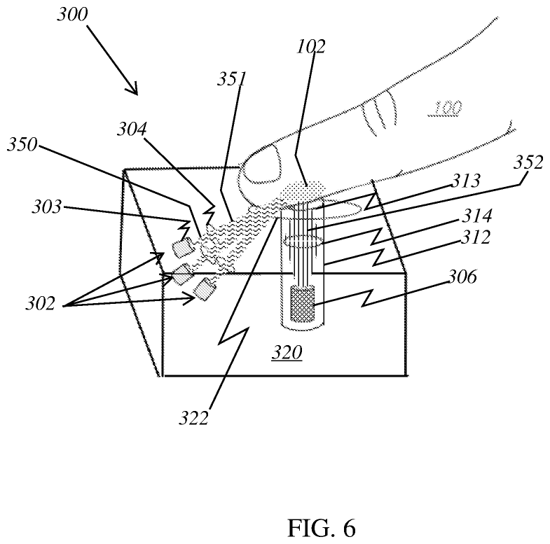

FIG. 6 schematically shows a RACAS 300 comprising a light shield for protecting a light sensor from stray light, in accordance with an embodiment of the disclosure.

In an embodiment RACAS 300 comprises at least one and optionally as schematically shown in FIG. 6 three or more light sources 302 for illuminating assay tissue of a person's finger with assay light and a light sensor 306 for sensing assay light reflected from the illuminating assay light by the assay tissue. Light sensor 306 is seated inside an optionally cylindrical light shield 312 formed from a material that is opaque to assay light and has an entry port 313 through which light may enter the light shield to reach sensor 306. Light entering light shield 312 through entry port 313 is optionally collected and directed to the light sensor by a collecting lens 314. The light sources, light sensor, and light shield are housed in a housing 320 having an access opening 322 on a top wall 324 of the housing. Light shield 312 is mounted inside housing 320 so that entry port 313 of the shield is optionally substantially within and coplanar with the access opening.

A person having BAC assayed by RACAS 300 places a finger to overlay access opening 322 and cover entry port 313 so that a region of the finger may be illuminated and provide reflected assay light incident on light sensor 306 as discussed below. Optionally access opening is covered by a protective cover (not shown) on which a person places a finger to have the person's BAC assayed. FIG. 6 schematically shows a finger 100 of a person having BAC assayed by RACAS 300 placed over entry port 313.

Optionally each light source 302 comprises at least one light emitting element 303 that emits assay light and an optical collimator lens 304 that receives light 350 from the light emitting element and collimates the light into a beam 351 of the assay light. In an embodiment all the light sources 302 are mounted in housing 320 so that beams 351 of assay light 350 from all the light sources propagate through access opening 322 and converge to substantially overlap and illuminate a same assay region 102 in finger 100. Optionally each light source comprises a focusing lens that focuses beam 351 to the assay region. Reflected assay light 352 reflected from beams 351 by assay tissue in assay region 102 enters light shield 312 through entry port 313 and is directed by collecting lens 314 to light sensor 306.

In an embodiment the at least one light emitting element 303 of a light source 302 comprises a plurality of light emitting elements (not shown in FIG. 6). The plurality of light emitting elements may comprise LEDs and/or laser diodes and provide assay light at a plurality of different assay wavelengths. Optionally each light source 302 provides assay light at 1300 nm, 1460 nm, and 2300 nm wavelengths.

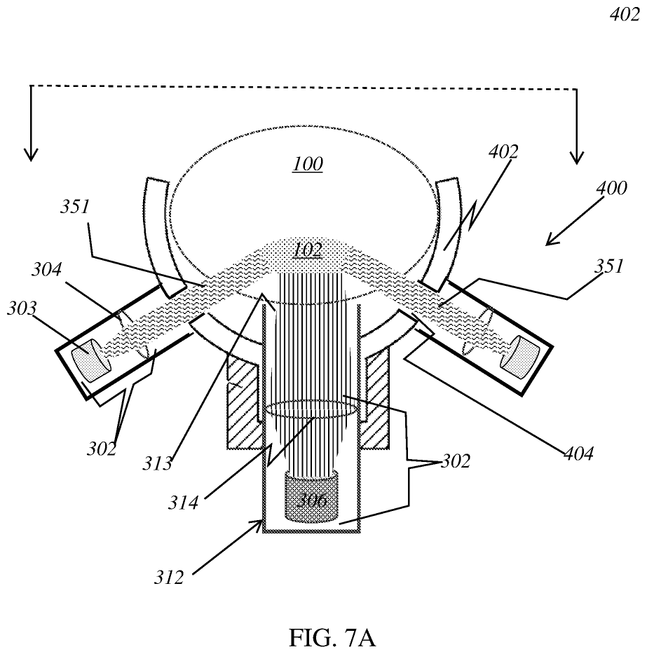

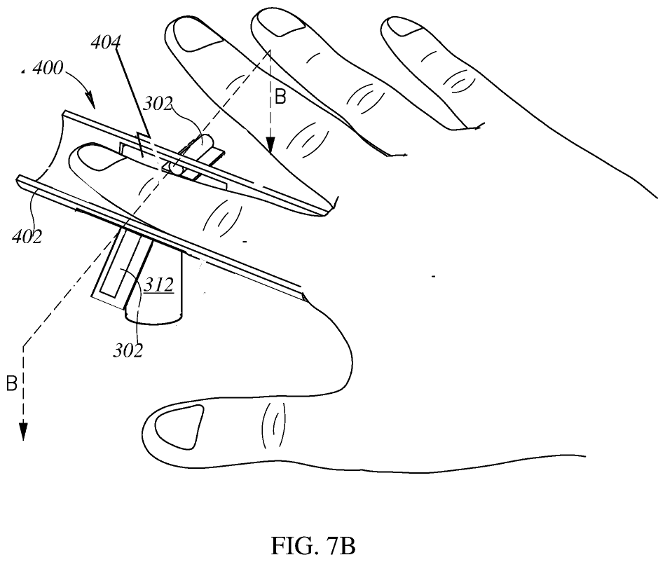

FIGS. 7A and 7B schematically show cross-section and perspective views of a RACAS 400, in accordance with an embodiment of the disclosure. The cross section is in a plane indicated as plane BB in both FIGS. 7A and 7B.

RACAS 400 comprises a finger cradle 402 for receiving a finger 100 of a person having BAC assayed by the RACAS, in accordance with an embodiment of the disclosure. In an embodiment RACAS 400 comprises at least one and as schematically shown in FIGS. 7A and 7B two light sources optionally similar to light source 302 shown in FIG. 6. The light sources are mounted to cradle 402 so that light beams 351 from the light sources pass through access openings 404 formed in the cradle to converge and illuminate a same assay region 102 in finger 100. A light sensor shielded by a light shield, optionally similar to light sensor 306 and shield 312 respectively shown in FIG. 6 is mounted to cradle 402 below where cradle 402 receives finger 100 to receive assay light reflected from assay tissue in assay region 102. Optionally access opening 404 and entry port 313 are covered by a protective cover (not shown) on which a person places a finger to have the person's BAC assayed by RACAS 400.

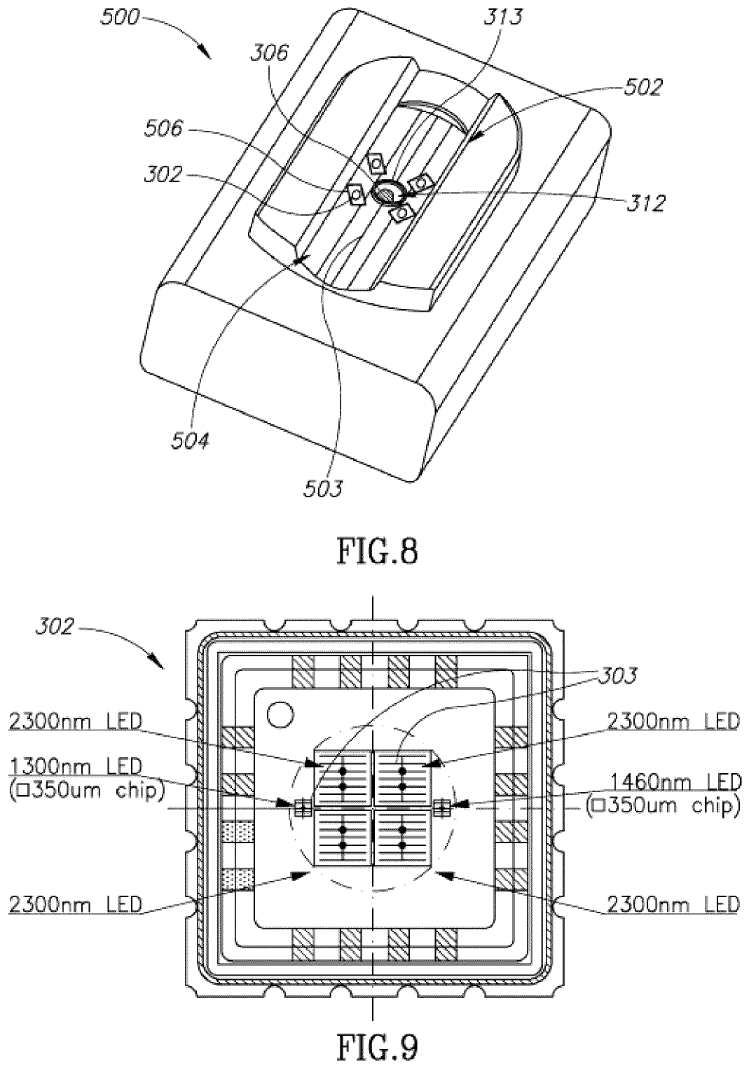

FIG. 8 schematically shows yet another RACAS 500 comprising a shielded light sensor in accordance with an embodiment of the disclosure.

RACAS 500 optionally comprises a finger cradle 502 a light sensor shielded by a light shield, which may be similar to light sensor 306 and shield 312 respectively shown in FIG. 6 and optionally a plurality of four light sources 302. Shield 312 has an entry port 313 located optionally on a bottom region 503 of a surface 504 of the cradle. Light sources 302 are located and positioned so that assay light from the light sources pass through respective exit windows 506 on surface 504 to illuminate a same region of a finger placed in the cradle in contact with entry port 313 and exit windows 506 or a protective cover overlying the entry port and exit windows.

FIG. 9 shows a schematic of a light source 302 comprising a configuration of a plurality of light emitting elements 303 that emit assay light, in accordance with an embodiment of the disclosure.

There is therefor provided in accordance with an embodiment, apparatus for assaying blood alcohol concentration (BAC), the apparatus comprising: a housing for receiving a finger of a person; a at least one light source that transmits light at at least one assay wavelength in respective directions so that the transmitted light from all of the at least one light source converges to overlap in a same convergence region that illuminates an assay region of a finger received by the housing; a light sensor positioned to receive and generate an assay signal responsive to assay light reflected by tissue in the assay region; and a light shield that enables assay light from the convergence region to reach the light sensor and shields the light sensor from stray light that is not reflected by tissue in the assay region from assay light originating from the at least one light sources. Optionally, the light shield comprises a tube surface that defines a lumen and an entrance port facing the convergence region through which light may pass to enter the lumen. Optionally, the light shield houses a collecting lens that receives light that enters the lumen through the entrance port and directs the light to the sensor. Optionally, the light sensor is located in the lumen.

In an embodiment the light from each of the at least one light source passes by the entrance port of the light shield when propagating to the convergence region. In an embodiment the at least one light source comprises a plurality of light sources all which are located on a same side of the convergence region. In an embodiment the at least one light source comprises a plurality of light sources at least two of which are located on opposite sides of the convergence region.

In an embodiment the housing comprises a cradle having a surface for receiving the finger. Optionally, each of the at least one light source transmits assay light through an exit window located on the surface of the cradle. Optionally, the exit window each of the at least one light source is covered by a protective cover transparent to assay light. In an embodiment the light shield entrance port is located on the surface of the cradle. Optionally, the entrance port is covered by a protective cover transparent to assay light.

In an embodiment the apparatus comprises a controller that processes the assay signal from the light source to determine a value for the BAC.

Descriptions of embodiments of the invention in the present application are provided by way of example and are not intended to limit the scope of the invention. The described embodiments comprise different features, not all of which are required in all embodiments of the invention. Some embodiments utilize only some of the features or possible combinations of the features. Variations of embodiments of the invention that are described, and embodiments of the invention comprising different combinations of features noted in the described embodiments, will occur to persons of the art. The scope of the invention is limited only by the claims.

1. Apparatus for assaying blood alcohol concentration (BAC), the apparatus comprising:
a housing for receiving a finger of a person;
at least one light source that transmits light at at least one assay wavelength in respective directions so that the transmitted light from all the at least one light source converges to overlap in a same convergence region that illuminates an assay region of a finger received by the housing;
a light sensor positioned to receive and generate an assay signal responsive to assay light reflected by tissue in the assay region; and
a light shield that enables assay light from the convergence region to reach the light sensor and shields the light sensor from stray light that is not reflected by tissue in the assay region from assay light originating from the at least one of light source.

2. The apparatus according to claim 1 wherein the light shield comprises a tube surface that defines a lumen and an entrance port facing the convergence region through which light may pass to enter the lumen.

3. The apparatus according to claim 2 wherein the light shield houses a collecting lens that receives light that enters the lumen through the entrance port and directs the light to the sensor.

4. The apparatus according to claim 3 wherein the light sensor is located in the lumen.

5. The apparatus according to claim 2 wherein the light from each of the at least one light source passes by the entrance port of the light shield when propagating to the convergence region.

6. The apparatus according to claim 1 claims wherein the at least one light source comprises a plurality of light sources all which are located on a same side of the convergence region.

7. The apparatus according to claim 1 wherein the at least one light source comprises a plurality of light sources at least two of which light sources are located on opposite sides of the convergence region.

8. The apparatus according to claim 1 wherein the housing comprises a cradle having a surface for receiving the finger.

9. The apparatus according to claim 8 wherein each of the at least one light source transmits assay light through an exit window located on the surface of the cradle.

10. The apparatus according to claim 9 wherein the exit window of each of the at least one light source is covered by a protective cover transparent to assay light.

11. The apparatus according to claim 8 wherein the light shield entrance port is located on the surface of the cradle.

12. The apparatus according to claim 11 wherein the entrance port is covered by a protective cover transparent to assay light.

13. The apparatus according to claim 1 and comprising a controller that processes the assay signal from the light source to determine a value for the BAC.
